(19) United States
(12) Patent Application Publication
(10) Pub. No.: US 2026/0137815 A1
(43) Pub. Date: May 21, 2026

(54) DIAGNOSTIC AND TREATMENT METHODS FOR ONYCHOMYCOSIS

(71) Applicant: ToeFX Inc., Hamilton (CA)

(72) Inventors: Showkat Monika Yazdanian, Hamilton (CA); Irit Itzhaki Van-Ham, Vaughan (CA)

(21) Appl. No.: 18/705,897

(22) PCT Filed: Jul. 28, 2023
(86) PCT No.: PCT/CA2023/051025

§ 371 Date: Apr. 29, 2024

Related U.S. Application Data
(60) Provisional application No. 63/393,110, filed on Jul. 28, 2022.

(51) Int. Cl. A61K 49/00; A45D 29/00; A61B 5/00; A61B 17/54; A61K 9/00; A61K 41/00; A61P 31/10; G06T 7/00; G06T 7/90; G06V 10/764; G06V 10/82

(52) U.S. Cl. CPC A61K 49/006; A45D 29/00; A61B 5/0071; A61B 17/54; A61K 9/0012; A61K 41/008; A61P 31/10; G06T 7/0014; G06T 7/90; G06V 10/764; G06V 10/82; A61B 2505/05; G06T 2207/10024; G06T 2207/20084; G06T 2207/30088; G06V 2201/03

(57) ABSTRACT

Diagnostic and treatment methods for onychomycosis are disclosed. The diagnostic method includes applying a Methylene Blue composition to nails suspected of onychomycosis infection and visually assessing the methylene blue staining on the nail following a diagnosis waiting period, as an indication of onychomycosis infection and severity. The severity of infection may be assessed qualitatively by comparison against a color guide and/or assessed quantitively by UV-Visual spectroscopy and correlating the spectral peak intensity and/or spectral shift to a quantitative onychomycosis severity scale. The diagnostic method may be combined with the photosensitizer activity of Methylene Blue to further provide a photodynamic regimen for treating onychomycosis, and a maintenance regimen for preventing re-infection of the nail.

### TECHNICAL FIELD

The embodiments disclosed herein relate to diagnosis and treatment of fungal infection, and, in particular to a method for diagnosis and treatment of onychomycosis.

### INTRODUCTION

Onychomycosis is an infection of the nail plate, nail bed, and, in some cases, the nail matrix and skin surrounding the nail plate, typically caused by fungal species such as T. rubrum. Onychomycosis can cause pain, discomfort, inflammation and disfigurement and may produce serious physical and occupational limitations, as well as reducing quality of life. The prevalence of onychomycosis among the general population is reported to be as high as 23% across Europe, 20% in East Asia, and 14% in North America, with an even greater predisposing risk factor in the adults over 60 years old.

Referring to FIG. 1, shown therein is a diagram of a human nail 100 (i.e., a toenail or a fingernail). Infecting fungi typically feed on keratin, a protein that forms a key structural component of the nail 100. The primary site of the infection is the nail bed 102, where the acute infection occurs with a low-grade inflammatory response and progresses to a chronic phase of the nail bed 102 infection as total dystrophic onychomycosis. Histologically, the acute onychomycosis infection manifests as spongiosis, acanthosis, papillomatosis with edema, and hyperkeratosis. These signs resemble the pathology of psoriasis. As in most infections, a dense inflammatory infiltrate develops.

Onychomycosis secondarily damages the nail germinator matrix 104 as the nail bed 102 becomes hyperkeratotic and thickened in an effort to shed the fungal infection. The fungus also invades the overlying nail plate 106, detaching and distorting it over time. The nail plate 106 becomes elevated and misaligned as the infection enters the chronic total dystrophic clinical stage of onychomycosis (TDO).

Treatments for onychomycosis are segmented into oral and topical applications, both with limiting factors. Oral administration, such as terbinafine, itraconazole, and fluconazole, has been available for many years, but their use is limited by a narrow spectrum, long courses, high relapse rates, and possible risks of hepatotoxicity. As for topical administration, topical products include Penlac® ciclopirox nail lacquer, which shows an underwhelming complete cure rate between 5.5% to 8.5%, as well as Jublia® efinaconazole and Kerydin® tavaborole, which show slightly higher complete cure rates of 15%-18% and 6.5%-9%, respectively. The limiting factors of available drugs have resulted in an unmet demand for onychomycosis treatment for safer, cheaper, and more effective and convenient treatments. Due to the persistence and resilience of the causative fungal organisms, onychomycosis treatments typically require multiple applications, administered over the course of months.

A further problem is, obtaining an accurate diagnosis of onychomycosis infection, as well as an indication of the progress or efficacy of a treatment course, is challenging. This problem has been described at length in the literature (see for example, Archana Singal et al, Onychomycosis: Diagnosis and Management, Symposium-Nails Part 1, 77:6, pp. 659-672, 2011). A visual appearance of onychomycosis may provide some indication of the particular infection, however, other modalities such as psoriasis and nail dystrophy may manifest with a similar presentation. It is important to confirm onychomycosis before initiating treatment, so that the correct treatment can be administered. Thus, a properly confirmed diagnosis of onychomycosis is paramount for the success of the treatment.

To date, a successful diagnosis of the infection, and the choice of treatment, critically depend on proper sample collection. A standard method for diagnosis entails taking a nail scraping, searching for fungal filaments under a microscope and taking a culture (Grover, C. et al, Journal of Dermatology, 2003, 30(2), 116-122). Samples of the specimens must be stored under the appropriate conditions that prevent rapid multiplication of microbial organisms and must be processed within two weeks to prevent false diagnosis. Even with these precautions, culture is not a robust diagnostic and is known to yield a relatively high false negative rate. Moreover, neither culture nor microscopic examination of fungal filaments can be used to quantify the severity of an infection, nor to assess the fungal load. These methods confirm or deny the presence of a fungal pathogen but do not afford any insights about efficacy of a treatment course.

A class of molecules called photosensitizers generate cytotoxic species, particularly singlet oxygen, when irradiated with light of an appropriate wavelength. Upon absorbing a photon, the photosensitizer molecule is promoted to a higher energy state, which then transfers its energy to an oxygen molecule, resulting in the generation of singlet oxygen. This phenomenon can be used to kill fungi and bacteria in a process called “lethal photosensitization” in which the reactive oxygen singlet damages bacterial and fungal membranes and DNA. Singlet oxygen has a very short lifetime and must be generated in close proximity to cells to produce the cytotoxic effects.

Photodynamic therapy (PDT) is a known method of disinfecting or sterilizing a hard or soft tissue site by topically applying a photosensitizing compound to the site, and then irradiating this with light at a wavelength that is absorbed by the photosensitizing compound, so as to destroy microbes at the site. PDT has been used for the treatment of various health conditions, such as certain cancers of the esophagus and lung, as well as actinic keratosis (AK), a skin condition that can develop into cancer.

The application of PDT in dentistry and medicine are wide and include the destruction of bacterial, fungal, and viral pathogens in a range of clinical settings. An antimicrobial photosensitive composition and a method of photodynamic therapy for the treatment of yeast, fungal, bacterial, and viral skin, wart, nail, and tropical infections are disclosed in the U.S. Patent Publication No. US20210046137A1, to the same inventor, which is incorporated into this disclosure in its entirety.

Thus, there is a need for an in-vivo method for diagnosis of onychomycosis quickly and accurately without use of culture. There is a further need for a preventative regimen that gradually reduces the onychomycosis infection and preferably eradicates it, and further prevents the re-infection of the affected nails.

### SUMMARY

According to an embodiment, there is a diagnostic method for onychomycosis. The method comprises applying a composition containing 0.01-10% v/v Methylene Blue onto the nail, waiting for the composition to penetrate into the nail and selectively stain regions of dermatophytic onychomycosis in vivo, and visually assessing Methylene Blue staining on the nail as an indication of onychomycosis infection, severity and treatment progress. The nail may be a toenail or a fingernail.

Visually assessing Methylene Blue staining may comprise qualitatively assessing the surface distribution and color of the Methylene Blue staining on the nail as an indication of the severity of onychomycosis, by comparison against a reference guide. Visually assessing Methylene Blue Staining may comprise conducting a UV-Visual spectroscopic measurement of the nail surface either in situ or in an extracted sample of nail and correlating the spectral shift to a quantitative onychomycosis severity score. The method may also entail calculating a ratio of stained regions of the nail to unstained regions of the nail, as an indication of the severity or extent of the infection. If this ratio is measured at regular intervals during the treatment course, it may be used as a method of assessing the efficacy of treatment.

The diagnostic method may further comprise mechanically thinning the nail to facilitate penetration of the composition into the nail and/or micro drilling holes in the nail to promote penetration of the composition into a nail bed.

In another embodiments, the diagnostic method, when combined with the photosensitizer activity of methylene blue, provides 1) a photodynamic regimen for treating onychomycosis after confirming presence of the infection and 2) a maintenance regimen to keep the infection at bay or prevent re-infection of the nail. It is preferable for the diagnostic method to precede the photodynamic therapy and maintenance regimens.

Other aspects and features will become apparent, to those ordinarily skilled in the art, upon review of the following description of some exemplary embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings included herewith are for illustrating various examples of articles, methods, and apparatuses of the present specification. In the drawings:

FIG. 1 is a diagram of the anatomy of a human nail;

FIG. 2 is a flow chart of a diagnostic method for onychomycosis, according to an embodiment;

FIG. 3 is a table of onychomycosis infection severity, according to an embodiment;

FIG. 4 is a representative image of a patient's toenails immediately after application of a Methylene Blue composition, according to an embodiment;

FIG. 5A is a plot of the absorption and emission spectra for Methylene Blue;

FIG. 5B is a plot of the absorption spectra for Methylene Blue when incubated with T. rubrum over time, according to an embodiment;

FIG. 5C is a plot of the absorption spectra for Methylene Blue when incubated with varying concentrations of T. rubrum, according to an embodiment;

FIG. 5D is a plot of absorption spectra for T. rubrum in culture, according to an embodiment;

FIG. 6 is a of a method for combined diagnosis and photodynamic therapy of onychomycosis, according to an embodiment;

FIG. 7A is representative images of Methylene Blue staining on nails infected with onychomycosis;

FIG. 7B is representative images of Methylene Blue staining on healthy nails;

FIGS. 8A-8B are representative images of nails showing Methylene Blue selectively staining localized to regions with onychomycosis; and

FIG. 9 is a representative image of Methylene Blue staining for guiding micro drilling of holes.

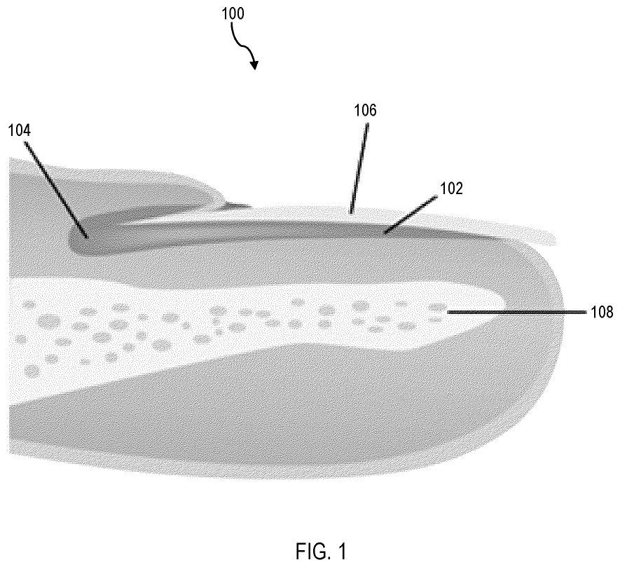

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

References herein to “MB” means Methylene Blue. Methylene Blue is a well-characterized, fluorescent, positively charged (cationic), phenothiazinium dye and photosensitizer. MB has known uses as an in-vitro fungal stain. MB in aqueous solution, preferentially stains microbes whose cell walls are negatively charged. The cell walls of gram-negative microorganisms, including onychomycosis-causing fungal cells, have higher lipid content than gram-positive cells and bear a negative charge, which has a strong affinity for positively charged molecules as opposed to mammalian cells, which do not. MB also displays acidophilic properties and may preferentially penetrate/intercalate into cells with increased amounts of nucleic acid, indicative of highly dysplastic and malignant cells that divide rapidly, like the fungal cells that give rise to onychomycosis.

### Diagnostic Methods for Onychomycosis

Photodynamic therapy (PDT) is effective in the treatment of onychomycosis infections, when applied according to described protocols, for example, as disclosed in U.S. Patent Publication No. US20210046137A1. Herein, it is described that PDT techniques are also effective in diagnosing DO infection, thereby providing for accurate diagnosis of the extent of infection for proper treatment of the disease.

MB under the name Loffler's Stain has been used as an in vitro tissue marker. MB has also been used in vivo for the detection of dental caries (infected dentin). Herein, a diagnostic method for onychomycosis using a MB composition to stain fungal cells in vivo is described.

The degree/extent/presence of the onychomycosis infection is assessed according to the degree of adsorption of the MB to the fungal organism. This adsorption can be assessed either qualitatively (visually) due to MB's vivid blue colour, e.g., by comparison against a reference guide or quantitatively by UV-Visual spectroscopic measurement of the MB staining of the nail surface, either in situ or from an extracted sample of the nail. The MB composition is preferably one of those disclosed in U.S. Patent Publication No. US20210046137A1 having MB between 0.01%-10% v/v, and preferably between 0.5-2% v/v in the composition.

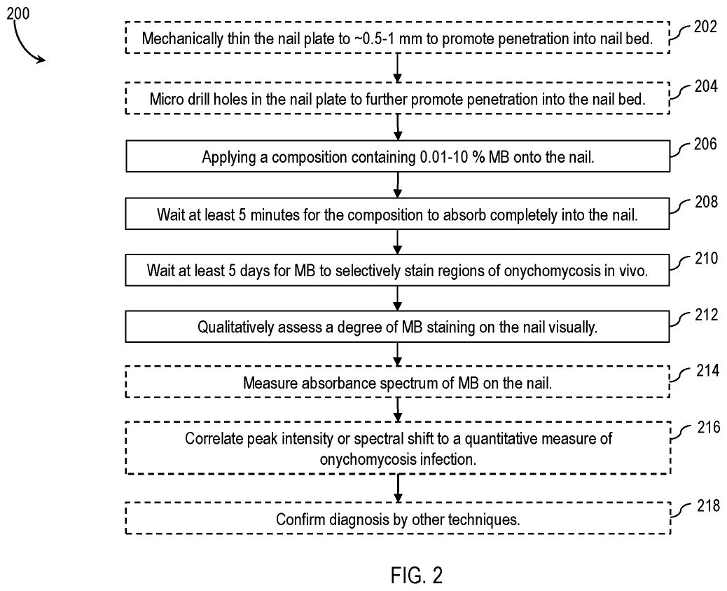

Referring to FIG. 2, shown therein is a flow chart of a diagnostic method 200 for onychomycosis. The diagnostic method 200 is aimed at the identification of dermatophytes (i.e., onychomycosis-causing microorganisms that require keratin for growth, as well as molds and yeast such as Candida Parapsilosis and Candida Guillermondii). The diagnostic method 200 is effective for identification of the infection in-vivo. In the absence of the diagnostic method 200, a patient with onychomycosis may not receive an appropriate treatment, or their condition may worsen as a result of the delay if onychomycosis is initially misdiagnosed. The diagnostic method 200 is preferred for diagnosis of onychomycosis, but may be adapted for diagnosis of other fungal infections, as will be understood by those skilled in the art.

At 202, a suspected infected nail is mechanically thinned, i.e., debrided to reduce the thickness of the nail plate. In some embodiments, dystrophic areas of the nail may be shaped into a channel to allow better penetration of the composition into the nail bed. As noted above, an infected nail displays certain physical characteristics such as inflammation, discoloration, dystrophy, etc. Nails infected with onychomycosis are also typically very thick compared to healthy nails. Accordingly, it is necessary to facilitate access to the nail bed by reducing the thickness of the nail plate so that a MB composition can penetrate the nail plate and flow to the nail bed where the infection mainly resides. Debriding the nail also mechanically removes fungal buildup on the keratin of the nail and further removes the hydrophobic protective layer of the nail. Nails will not require debriding if they are sufficiently thin.

Where necessary, the nail plate is debrided, using a standard nail drill, to a thickness of no more than 2 mm, preferably to a thickness of about 1 mm, and more preferably to a thickness of about 0.5 mm to promote access to the nail bed and allow better penetration of the MB composition into the nail bed. The nail drill should be fitted with a suitable burr (i.e., a rotary file) that creates minimal heat and nail dust which can be a conduit for spreading fungal particles to other (potentially healthy) toes or other individuals. To further reduce the spread of fungal particles, all nails, whether suspected to be infected or not, are swabbed/cleaned with ethanol prior to debriding. Following debriding, the thickness of the nail plate should be confirmed to be no more than about 0.5-1.0 mm using calipers, a ruler or by visual inspection of a trained technician.

Particular care is required for debriding the corners of the nail plate, as these are areas where onychomycosis can linger and give rise to re-infection. In cases where nail dystrophy is evident, the preferred approach to debriding the nail plate is to file or shape the dystrophic areas into a channel to allow better penetration into the nail bed.

Generally, only suspected infected nails should be debrided. However, in the case of a suspected severe infection of one or more nails, other apparently health nails on the same foot/hand should also be debrided for diagnosis as a precaution. If blood is released or if the patient feels discomfort, debriding should be stopped. If the nail plate or a portion thereof becomes detached from the nail bed, it should be removed.

At 204, in the case of a suspected severe infection, micro drilling into the nail plate may be performed to further promote penetration into the nail bed. Holes are drilled ˜2 mm apart in the nail plate using a microdrill burr of no more than 1 mm in diameter and 1 mm in depth (or with a stopper to limit the drill depth). Holes should be drilled as close as possible to the nail germinator matrix. Apparently healthy nails should not be drilled.

Debriding, alone, or in combination with micro drilling enhances penetration of the MB composition into the nail by creating micro-channels in the nail plate, thereby promoting penetration of the MB composition into the nail bed where the infection typically resides. This also provides for transonychial water loss, allowing for an immediate cosmetic improvement to the nail.

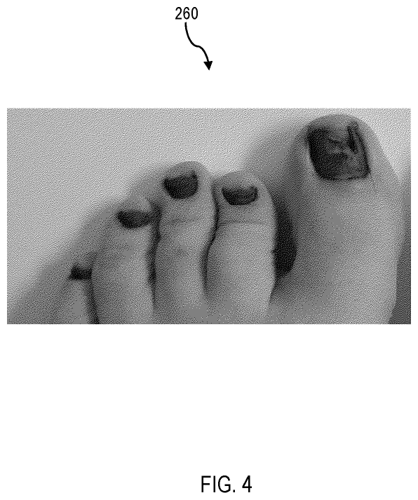

At 206, the surface of the suspected infected nail is saturated with the MB composition having MB in 0.01-10% v/v. FIG. 4 shows a representative image of a patient's toenails immediately after application of the MB composition. About 0.1-0.2 mL/cm² of the MB composition is applied onto the surface of the suspected infected nail; this translates into approximately 0.2 mL applied to the big nail (on the thumb or first toe) and approximately 0.1 mL applied to other nails. Where the nail plate has been drilled with holes, the MB composition should be dispensed directly through the holes and through the rest of the nail surface. The patient's foot/hand should be placed flat on a surface while the MB composition is dispensed onto the nail. According to other embodiments, application of the MB composition onto the nail can also be achieved via a drug-in-adhesive nail patch or transungual drug delivery system.

Due to the highly contagious nature of onychomycosis, all nails on the foot/hand should receive the MB composition, whether they appear to be infected or not. This is done to diagnose incipient infections on adjacent nails at an early stage when they may not yet produce visible damage to the nail (and are easier to treat). The composition should be dispensed onto each nail using a one-time use/disposable pipette, brush applicator or pre-filled syringe applicator that is replaced after applying to one nail to prevent cross-contamination between nails.

At 208, the MB composition is left on the nail for at least 5 minutes, and preferably longer, for the composition to be completely absorbed into the nail. For nails having a thickness greater than 1 mm, the composition should be left on the nail for longer to ensure complete absorption. The nail should be left uncovered during this time.

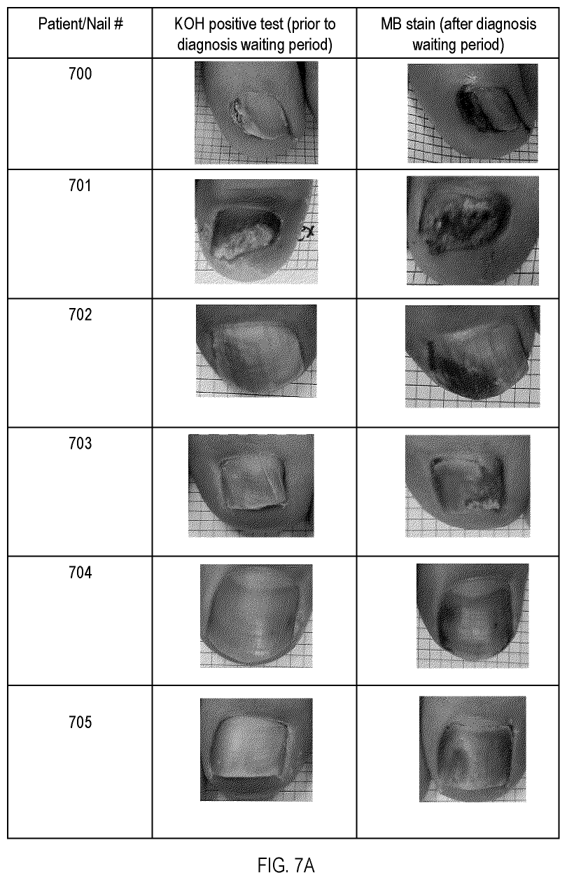

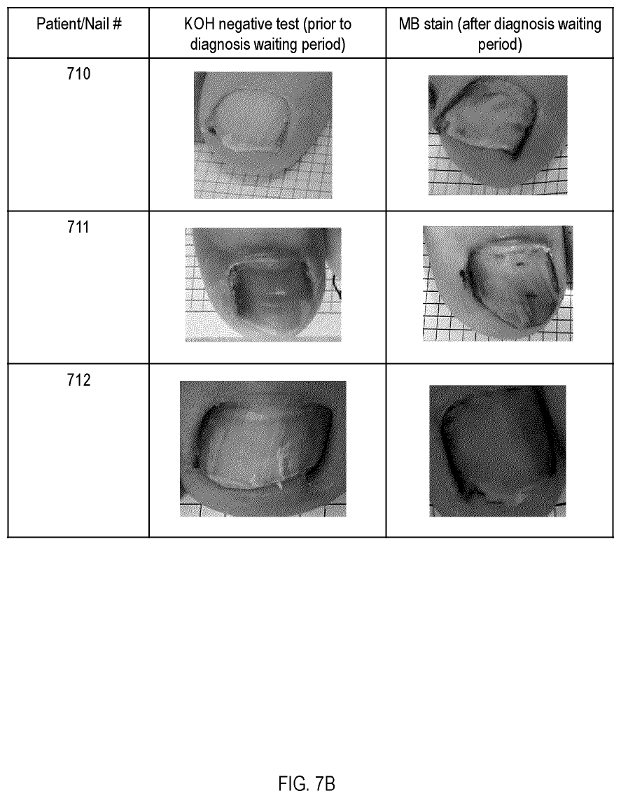

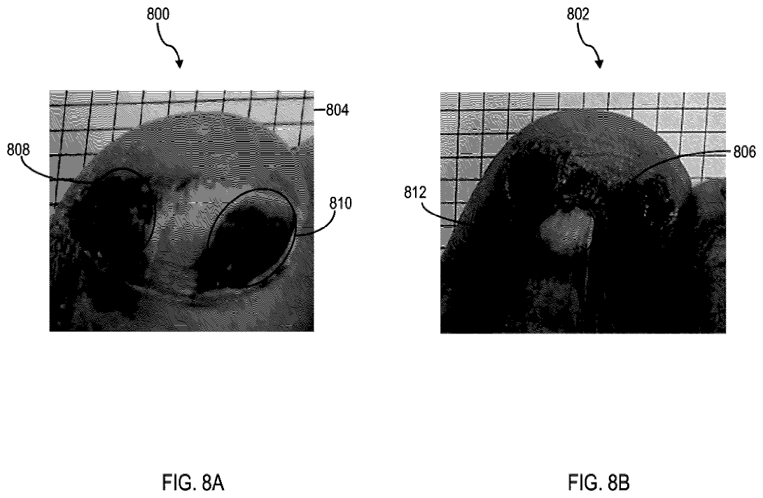

At 210, the MB composition is incubated on the nail for at least 5 days, and up to 14 days, the “diagnosis waiting period.” During the diagnosis waiting period, the MB in the composition is adsorbed into the nail matrix and selectively stains the microbes that cause onychomycosis, primarily dermatophytes (fungi that require keratin for growth) (see FIGS. 7-8). Dermatophytic fungal species are the most common causative agents of onychomycosis. Non-specific MB staining of skin cells on the nail will fade and not persist after the diagnosis waiting period; repeatedly washing the stained area with cleaning agents will accelerate fading. During the diagnosis waiting period, the nail, whether on foot or hand, can be covered by clothing (e.g., socks, shoes, gloves, etc.) or left open.

Referring to FIG. 7A, shown therein are representative images of onychomycosis infected nails 700, 701, 702, 703, 704, 705 wherein a positive KOH and culture test (using the protocol by Grover et al.) confirmed the presence of onychomycosis prior to application of the MB composition. Images of the same nails 700, 701, 702, 703, 704, 705 two weeks following the application of the MB composition to the nails (i.e., after the diagnosis waiting period) are also shown. As can be seen following the diagnosis waiting period, there is substantive surface distribution of MB staining retained on the nails 700, 701, 702, 703, 704, 705 visibly indicating the (continued) presence of onychomycosis after the diagnosis waiting period.

By contrast, referring to FIG. 7B, shown therein is representative images of healthy nails 710, 711, 712, wherein a negative KOH and culture test confirmed the absence of onychomycosis prior to application of the MB composition. Images of the same nails 710, 711, 712 two weeks following the application of the MB composition to the nails (i.e., after the diagnosis waiting period) are also shown. As can be there is minimal surface distribution of MB staining on the nails 710, 711, 712 after the diagnosis waiting period, and the staining that is visible is largely restricted to the periphery of the nails.

Referring to FIGS. 7A and 7B, when the MB composition is applied to a nail, MB selectively and strongly stains onychomycosis-infected nails blue whereas healthy nails will have minimal staining. A skilled technician will be able to differentiate between the infected nails 700, 701, 702, 703, 704, 705 and the healthy nails 710, 711, 712 by visibly observing the surface distribution of MB staining on the nail after the diagnosis waiting period.

Referring to FIGS. 8A-8B, shown therein are representative images 800, 802 of nails 804, 806 showing MB staining localized to regions with onychomycosis. The MB staining is localized to the specific regions of the nail where onychomycosis is present. Other nail conditions such as bruised nails and nail dystrophy are not stained.

Referring now to FIG. 8A, shown therein is a nail 804 that tested positive for onychomycosis in a KOH test. After applying the MB composition and waiting the diagnosis waiting period, a region of localized onychomycosis 808 is visible indicated by localized MB staining blue in color. The nail 804 also includes a region of blunt force trauma 810 indicated by discoloration. A skilled technician will be able to differentiate the blue MB-stained region of localized onychomycosis 808 from the discolored region of blunt force trauma 810.

Referring now to FIG. 8B, shown therein is a nail 806 afflicted with nail dystrophy. After applying the MB composition and waiting the diagnosis waiting period, no MB staining is visible on the dystrophic portion of the nail 812, whereas the rest of the nail 806 shows substantive surface distribution of MB staining indicating the presence of onychomycosis.

Referring again to FIG. 2, at 212, the degree of MB staining of fungal cells after completion of the diagnosis waiting period is assessed visually. Visually assessing MB staining may comprise qualitatively assessing the surface distribution and color of the MB staining on the nail as an indication of the severity of onychomycosis, by comparison against a reference guide. Visually assessing MB staining may entail calculating a ratio of stained regions of the nail to unstained regions of the nail, as an indication of the severity or extent of the infection. If this ratio is measured at regular intervals during a treatment course, it may be used as a method of assessing the efficacy of the treatment.

It should be noted that MB appears blue in color under ambient (white) light. Following the diagnosis waiting period, the infected areas of the nail will retain a blue color and display chipping and degradation due to the infection. By contrast, the blue color will fade away nearly completely from the surface of healthy, uninfected nails and there will be no evidence of chips or cracks.

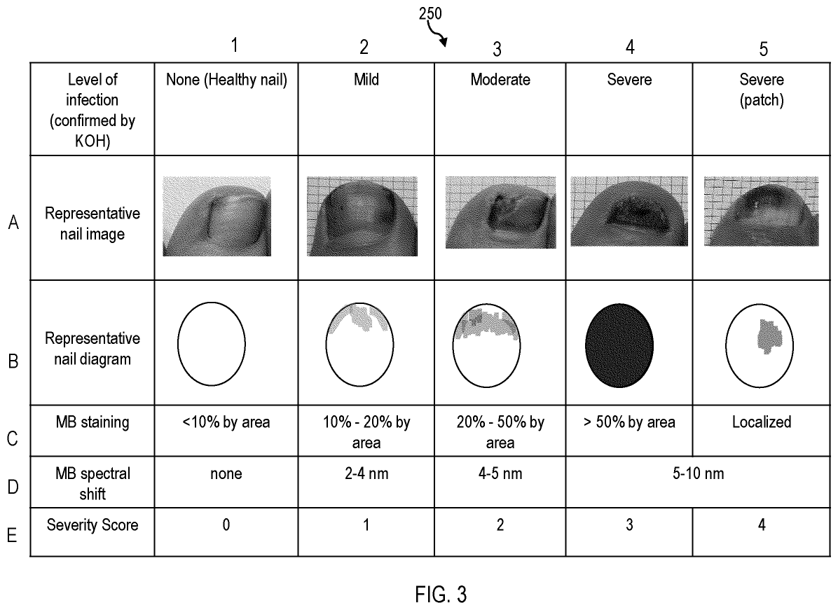

Referring to FIG. 3, shown therein is a table of onychomycosis infection severity 250. Row A shows representative images of nails (toenails) with varying levels of onychomycosis infection as confirmed by a positive KOH test and traditional culture techniques (Grover et al, 2014). Row B shows representative greyscale diagrams of the representative images in Row A. Row C shows the degree of MB staining on the nail as a percentage of the nail area. Row D shows the MB spectral shift caused MB absorption to fungal organisms (explained below). Row E shows an onychomycosis severity score indicating the degree of onychomycosis infection.

Cells A1 and B1 show a healthy nail i.e., without detectable infection. MB staining is present on <10% of the nail area and is mostly observed on the skin at the periphery of the nail, but little or no blue staining is present on the nail itself. The nail is healthy and appears shiny and reflective with no visible chipping or cracks.

Cells A2 and B2 shows a mild infection corresponding on a severity score of 1. Most of the nail is unaffected, but diffuse blue MB staining is present on 10-20% of the nail area. The infected portion of the nail may be slightly chipped and much of the nail looks healthy.

Cells A3 and B3 show a moderate infection. The nail becomes brittle and may show more chipping (compared to the mild infection) and 20%-50% of the nail area is infected and stained blue. The nail strongly resist attempts to purge the blue color e.g., by washing. The remainder of the nail looks healthy.

Cells A4 and B4 show a severe infection. Substantially the entire nail area (at least >50%) is stained dark blue and there is significant chipping and cracking across the entirety of the nail.

Cells A5 and B5 show a severe patch infection. The nail has an infected patch that is stained deep blue around the infected locus with significant chipping and cracking of the nail. A patch may be an indication of the presence of fungal spores which are typically more difficult to treat than mature fungal cells. Also, patches are thicker and extend deeper into the nail bed. It should be noted that patches may occur in mild and moderate infections. Generally, when an patch is present, the severity score in FIG. 3 should be increased by 1. For example, a mild infection will have a severity score of 1, whereas a mild patch infection will have a severity score of 2.

The color of the MB stain on the nail after the diagnosis waiting period may be compared against a reference guide e.g., a set of representative toenail images (e.g., row A in FIG. 3) for a qualitative assessment of the level of infection. The qualitative assessment may provide an indication whether a quantitative assessment of the level of infection should be performed. For example, if no nails show a qualitative level of infection, a subsequent quantitative assessment is not needed. If on the other hand one or more nails qualitatively show a level of infection, then a quantitative assessment of the level of infection is performed.

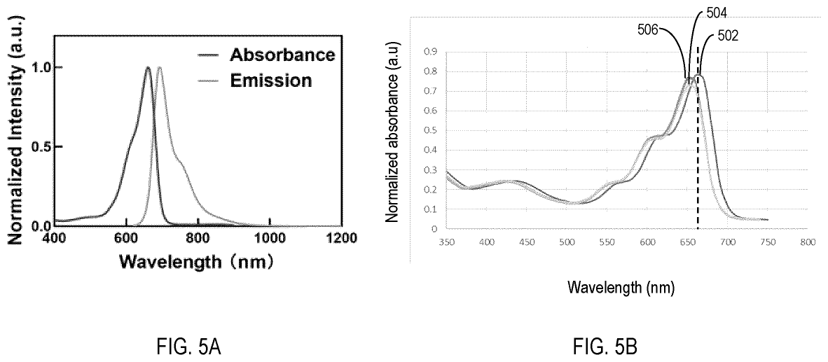

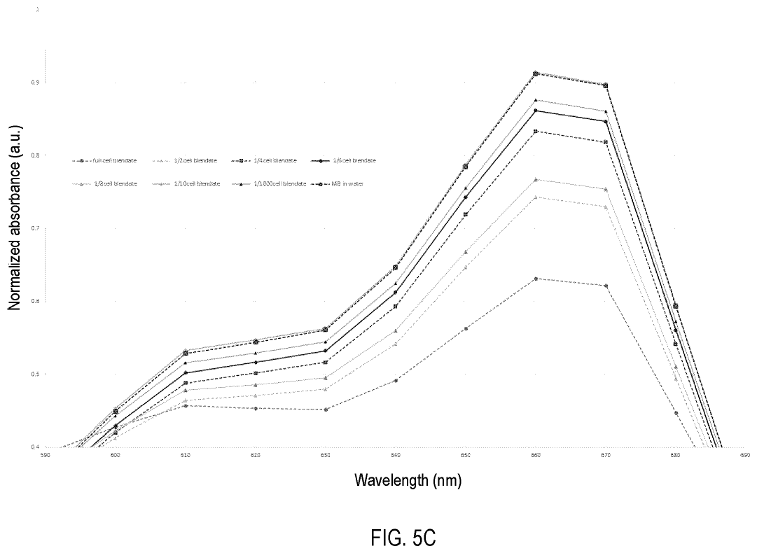

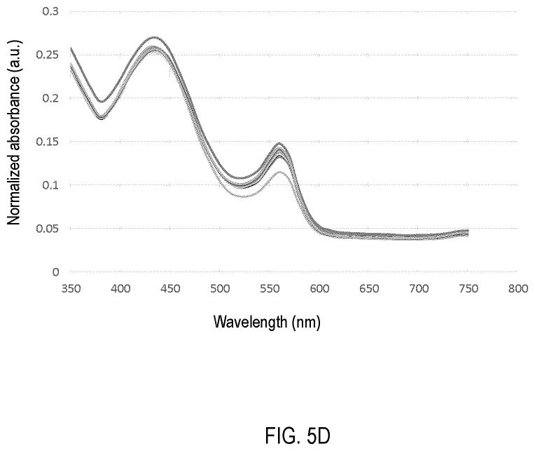

Referring again to FIG. 2, at 214, if the qualitative assessment at 212 indicates the presence of onychomycosis infection, the UV-Visual spectroscopic measurement of the MB staining of the nail, either in situ or from an extracted sample of the nail is performed to provide a quantitative assessment of the degree of infection. When excited by red light between 620-670 nm, MB shows an absorption peak at 665 nm and an emission peak at 685 nm (FIG. 5A). A change in intensity and shifting of these peaks is observed depending on the degree of adsorption of MB to fungal organisms e.g., T. rubrum (see FIGS. 5B-5C). Accordingly, the intensity and shift in the peaks can be used as a quantitative measure of onychomycosis infection as described below. It may also be possible to directly measure the absorbance spectral shift of the fungal organism itself as an indication of the level of infection. For example, the absorbance peak for T. rubrum (in solution) occurs at ˜430 nm with a secondary peak at ˜560 nm (FIG. 5D).

The nail is illuminated with red light in the 620-670 nm range and the absorbance or emission spectra of MB on the nail is measured using a detector. A spectrophotometer may be used for this purpose. Alternatively, a smartphone configured to emit and detect visible light and installed with an application configured to filter out ambient light may be used to measure the signal

At 216, the presence of a spectral shift in the absorbance peak or increase/decrease in peak intensity of MB from the nail is correlated to a fungal load or an onychomycosis severity score to quantify the severity of DO infection. A higher severity score indicates a higher severity of infection. FIG. 3 lists the nanometer spectral shift in the MB absorbance spectrum and the corresponding severity score in rows D and E, respectively, for each level of infection. A severity score of 1 or higher indicates a positive diagnosis of infection.

Referring to FIG. 5C, shown therein are UV-Visual absorption spectra of MB when incubated with T. rubrum. T. rubrum strain F4520 cubes were retrieved from −80° freezer, thawed at room temperature, and transferred to an Sabourand dextrose agar (SDA) plate, and distributed evenly on the SDA plate. The SDA plate was sealed by parafilm and moved into a 30° C. incubator until vigorous fungal growth was observed. Next, fungal mycelia were prepared, by transferring a 6.25 cm² culture from the SDA plate to a sterile blender cup and adding 200 mL sterile water. After 30 seconds of blending, varying amounts of blendate was pipetted into wells of a 96 well plate. 150 μL of MB solution and an amount of sterile water was added to each well to make to total volume the same in each well. The MB absorbance spectra was then measured by a photospectrometer.

FIG. 5C shows varying concentrations of T. rubrum in a 0.01% v/v solution of MB. The vertical axis shows normalized absorbance of MB in arbitrary units; the horizontal axis shows absorbed wavelengths. The more dilute the fungus, the higher the MB absorbance value (i.e., normalized absorbance), indicating higher amounts of free MB in the solution. With increasing fungal concentration, lower MB absorbance is observed, indicating adsorption of MB to the fungus.

FIG. 5B shows the time dependence of the MB absorption spectrum for a 0.25% v/v solution of MB with T. rubrum culture. A similar protocol was followed as described above for FIG. 5C, except that the concentration of T. rubrum in the solution was kept constant. MB absorbance was measured immediately (curve 502), 4 days (curve 504), and 7 days (curve 506) after mixing. Immediately after mixing, the MB absorbance peak is centered at ˜660 nm, corresponding to the absorption peak of MB alone in solution (see FIG. 5A). Over time, the intensity of the absorbance peak is lowered as MB is adsorbed to fungal cells, consistent with the curves in FIG. 5C. There is also an observable blue-shifting of the MB absorbance peak over time, compared to MB alone (for reference the dashed line at 665 nm in FIG. 5B indicates the absorbance peak for MB alone), indicating binding of the MB and fungal cells.

Referring back to FIG. 2, at 218, a potassium hydroxide (KOH) test to confirm the presence of mycelia may be performed to confirm the diagnosis of DO infection. A KOH test entails placing a nail clipping specimen on a microscope slide with a drop of 10% to 20% KOH solution, then allowing the mixture to sit for at least five minutes before viewing under a microscope. KOH digests the nail matrix, and a skilled technician will be able to identify the presence of mycelia under the microscope. A KOH test can be performed, for example, on a smartphone with a microscope attachment that magnifies cells by at least 40×.

If desired, a positive diagnosis can be confirmed with the following techniques known in the art: 1) an Al-based method for diagnosis of onychomycosis, whereby a neural network trained to classify images of fungal infection compares patient nail images suspected of infection against control and reference nail images (e.g., nail images in row A in FIG. 3 and FIGS. 7A-7B); 2) a quantitative polymerase chain reaction (qPCR) based method for diagnosis of onychomycosis; 3) antigen methods for diagnosis of onychomycosis; 4) nail dermoscopy or 5) conventional culture techniques.

### Photodynamic Therapy Methods for Onychomycosis

The diagnostic method 200 may be used as a standalone method for diagnosing and quantifying the level of DO infection in nails. The method 200 may further be used in 1) a photodynamic therapy regimen for treating onychomycosis and 2) a maintenance regimen to prevent re-infection of the nail.

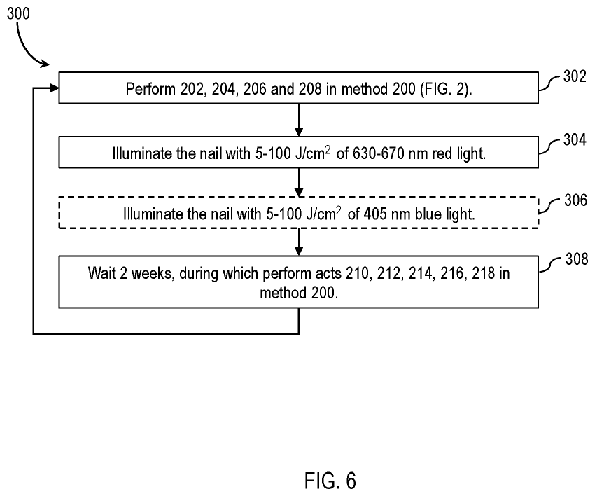

Referring to FIG. 6, shown therein is a flow chart of a method 300 for photodynamic therapy of onychomycosis, according to an embodiment. The method 300 may be used to treat DO infection of any severity.

At 302, acts 202, 204 (optional), 206 and 208 in method 200 are performed.

At 304, the nail is illuminated with a dose of 5-200 J/cm² of 630-670 nm red light. Illumination with red light activates MB as a photosensitizer to generate singlet oxygen which kills fungal cells by cytotoxicity. It should be noted that, at this point, most of the MB is “free” and not (yet) bound to fungal cells in the nail. The free MB is excitable by the red light to activate the MB photosensitizing activity. The power of the light dose is calculated as follows:

$$Power of light ( W / s \cdot {cm}^{2} ) \times time of exposure to light ( s ) = light dose ( J / {cm}^{2} )$$

At 306, the nail is illuminated with a dose of 5-200 J/cm² of 405-430 nm blue light to promote anti-inflammation and antimicrobial effects in infected nails. Act 306 is optional and may be performed before, after or simultaneously with act 304.

At 308, a 1-2-week period elapses during which acts 210, 212, 214, 216 and 218 in method 200 are performed. If there remains a positive diagnosis of infection after 2 weeks following acts 304, 306, then the method 300 is repeated until a negative diagnosis of infection is obtained, or the patient is satisfied with the appearance of the nail. During subsequent repetitions of the method 300, at step 302, if act 204 is performed, holes may be micro drilled in a region of the nail where Methylene Blue staining persists (indicating the continued presence of onychomycosis) to promote penetration of the MB composition to the nail bed in that region.

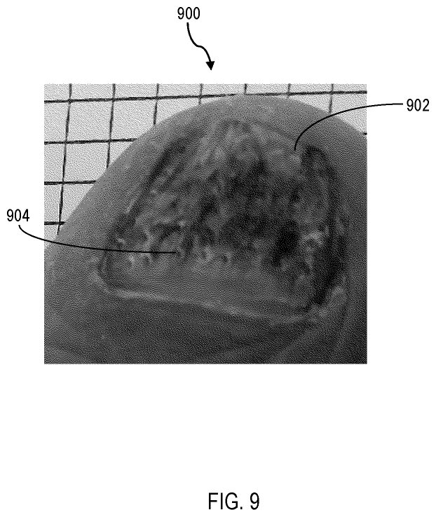

Referring to FIG. 9, shown therein is a representative image 900 of a nail 902 with MB staining for guiding micro drilling of holes 904. After the diagnosis waiting period, if MB stain persists on the nail 902 indicating the continued presence of onychomycosis, a clinician may drill sub-millimeter holes 904 using the MB stain as a guide to better facilitate penetration of the MB composition into the nail bed in the region of MB staining when the MB composition is subsequently reapplied to the nail 902. No holes are drilled in the uninfected region of the nail 902 (the region lacking MB stain) to minimize unintended damage to the nail matrix caused by drilling.

The method 300 is particularly advantageous compared to existing photodynamic methods of treatment for onychomycosis using topical treatments such as efinaconazole. Existing methods typically require daily application of a topical photosensitizer and illumination with light, which can be inconvenient for patients and difficult to adhere to, resulting in a lower success rate for treating the infection. The method 300 requires only bi-weekly application of the MB composition to the nail and illumination with light, thereby promoting adherence to the treatment regimen, and increasing the success rate for treating the infection.

Onychomycosis infections caused by dermatophytes are extremely persistent. After the patient is satisfied with the appearance of the nail, or there is a negative diagnosis of fungus, the focus of the treatment shifts to “maintenance”. Instead of administering the treatment every 1-2 weeks, a treatment is administered every 4-6 weeks. Clinical trial data has shown that maintenance treatments using the MB composition described herein, at a frequency of about every 4-6 weeks, over a period of 6 months, are effective at preventing the infection from worsening and the infection spread can be kept at bay.

While the present disclosure describes the use of MB as a stain to qualitatively and quantitatively assess the extent of onychomycosis, those skilled in the art will understand other dyes/stains with similar fungal or microbe staining properties that may be used in place of MB with the appropriate calibrations made to the reference guide for qualitative assessment of onychomycosis infection and the spectral peak intensity and spectral shift correlation to the onychomycosis severity score for the particular dye/stain.

Other suitable fluorophores may include Gentian violet, Porphyrins, chlorins, pheophorbides, bacteriopheophorbides, texaphyrins, and phthalocyanines. Suitable non-porphyrins fluorophores may include anthraquinones, phenothiazines, xanthenes, cyanines, curcuminoids, phthalocyanine, phenothiazinium, benzoporphyrins, haematoporphyrin, pyrrole, tetrapyrrolic compound, pyrrolic macrocycle, porfimer, 5-aminolevulinic acid, furanone, quinine, resorcinol, and terpenoids.

While the above description provides examples of one or more apparatus, methods, or systems, it will be appreciated that other apparatus, methods, or systems may be within the scope of the claims as interpreted by one of skill in the art.

1. A diagnostic method for onychomycosis, comprising:
applying a composition containing 0.01-10% v/v Methylene Blue onto a nail;
waiting for the composition to penetrate into the nail;
waiting between 5-14 days for the Methylene Blue to selectively stain regions of onychomycosis in vivo; and
visually assessing Methylene Blue staining on the nail as an indication of onychomycosis infection.

2. The diagnostic method of claim 1, further comprising:
calculating a ratio of Methylene Blue stained regions of the nail to unstained regions of the nail, as a qualitative indication of the severity or extent of the onychomycosis infection.

3. The method of claim 1, wherein visually assessing Methylene Blue staining comprises:
qualitatively assessing the surface distribution and color of the Methylene Blue staining by comparison against a reference guide.

4. The method of claim 1, further comprising:
UV-Visual spectroscopic measurement of the nail in situ or an extracted sample of the nail; and
correlating an intensity of Methlyene Blue spectral peaks to a fungal load or an infection severity score. The method of claim 1, further comprising:
UV-Visual spectroscopic measurement of the nail in situ or an extracted sample of the nail; and
correlating a spectral shift in Methylene Blue absorbance or emission to a fungal load or an infection severity score. The method of claim 5, wherein measuring the UV-Visual spectroscopic measurement comprises:
illuminating the nail with 630-670 nm red light; and
detecting absorbance or emission of Methylene Blue. The method of claim 1, further comprising:
mechanically thinning the nail or shaping dystrophic areas of the nail into a channel to facilitate penetration of the composition into a nail bed. The method of claim 1, further comprising:
micro drilling holes in the nail to promote penetration of the composition into a nail bed. The method of claim 1, wherein waiting for the composition to penetrate into the nail comprises:
waiting at least 5 minutes.

10. The method of claim 1, wherein the nail is a toenail or a fingernail.

11. The method of claim 1, further comprising:
UV-Visual spectroscopic measurement of the nail in situ or an extracted sample of the nail; and
detecting absorbance of T. rubrum at ˜430 nm.

12. The method of claim 1, further comprising:
confirming the presence of onychomycosis by one of:
a potassium hydroxide (KOH) test for mycelia or Arthroconidia;
a quantitative polymerase chain reaction (qPCR) based method for diagnosis of onychomycosis and rating its severity;
an antigen method for diagnosis of onychomycosis;
nail dermoscopy; and
conventional culture techniques.

13. The method of claim 1, further comprising confirming the presence of onychomycosis by an AI-based method, whereby a neural network trained to classify images of fungal infection compares images of the nail against control and reference nail images.

14. A method for combined of diagnosis and treatment of onychomycosis, comprising:
applying a composition containing 0.01-10% v/v Methylene Blue onto a nail;
waiting for the composition to penetrate into the nail;
waiting between 5-14 days for the Methylene Blue to selectively stain regions of onychomycosis in vivo;
visually assessing Methylene Blue staining on the nail as an indication of onychomycosis infection and severity; and
illuminating the nail with a dose of 5-200 J/cm² of 630-670 nm red light;

15. The method of claim 14, wherein the method is repeated every 7-14 days or every 4-6. weeks.

16. The method of claim 15, further comprising:
micro drilling holes in a region of the nail previously stained by Methylene Blue to promote penetration of the composition into a nail bed.

17. The method of claim 14, wherein waiting for the composition to penetrate into the nail comprises:
waiting at least 5 minutes.

18. The method of claim 14, further comprising:
illuminating the nail with a dose of 5-200 J/cm² of 405-430 nm blue light.

19. The method of claim 14, wherein visually assessing Methylene Blue staining comprises:
qualitatively assessing the surface distribution and color of the Methylene Blue staining by comparison against a reference guide.

20. The method of claim 14, further comprising:
UV-Visual spectroscopic measurement of the nail in situ or an extracted sample of the nail; and
correlating an intensity of Methylene Blue spectral peaks to a fungal load or an infection severity score.

21. The method of claim 14, further comprising:
UV-Visual spectroscopic measurement of the nail in situ or an extracted sample of the nail; and
correlating a spectral shift in Methylene Blue absorbance or emission to a fungal load or an infection severity score.
